(19) United States
(12) Patent Application Publication
(10) Pub. No.: US 2008/0020070 A1
(43) Pub. Date: Jan. 24, 2008

(54) Citrus Fruit Skin Extract for Angiogenesis Promotion

(75) Inventors: Paul Frank Davis, Wellington (NZ); Lai Yeap Foo, Wellington (NZ)

Correspondence Address: KNOBBE MARTENS OLSON & BEAR LLP, 2040 MAIN STREET, FOURTEENTH FLOOR, IRVINE, CA, 92614, US

(73) Assignees: Industrial Research Limited, Lower Hutt (NZ); University of Otago, Dunedin (NZ); Otago Innovation Limited, 481 Moray Place, Dunedin (NZ)

(21) Appl. No.: 11/587,716

(22) PCT Filed: Apr. 29, 2005
(86) PCT No.: PCT/NZ05/00084

§ 371 Date: Aug. 7, 2007

(30) Foreign Application Priority Data
Apr. 30, 2004 (NZ) 532666

(51) Int. Cl. A61K 36/752; A61P 7/00; C12N 5/06; C12N 5/08

(52) U.S. Cl. 424/736; 435/375

(57) ABSTRACT

The use of a citrus fruit skin extract for promoting angiogenesis, or treating a disease or disorder where it is desirable to promote angiogenesis. Such diseases include coronary heart disease, stroke, or ulcers. A composition comprising an extract of citrus fruit skin suitable for use in promoting angiogenesis, or treating a disease or disorder where it is desirable to promote angiogenesis. A process for preparing a citrus fruit skin extract. The citrus fruit may be grapefruit, orange, lemon, lime, mandarin, tangelo, tangerine or uglis, preferably grapefruit (Citrus paradisii).

### TECHNICAL FIELD

This invention relates to the use of a citrus fruit skin extract for promoting angiogenesis. In particular, the invention relates to the use of a grapefruit skin extract for treating diseases or disorders where it is desirable to promote angiogenesis, such as coronary heart disease, stroke or ulcers, and includes the assistance of wound healing.

### BACKGROUND

Angiogenesis is the growth and proliferation of new blood vessels. Angiogenesis plays an important role in many physiological and pathological conditions. In a healthy body, angiogenesis occurs during the proliferative phase of wound healing, for restoring blood flow to tissues after injury or insult, and in females during the monthly reproductive cycle and during pregnancy.

The healthy body controls angiogenesis through a series of “on” and “off” switches. The “on” switches are known as angiogenesis stimulating growth factors and examples include fibroblast growth factor (FGF), vascular endothelial growth factor (VEGF) and interleukin-8 (IL-8). In contrast, the “off” switches are called angiogenesis inhibitors. Examples of angiogenesis inhibitors include thrombospondin-1, angiostatin, and metallo-proteinase inhibitors. In the normal healthy body, the expression of angiogenesis growth factors and angiogenesis inhibitors is finely balanced in order to facilitate activation and suppression of angiogenesis.

Loss of control of angiogenesis can lead to many serious disease states. Angiogenesis-dependent diseases result when there is excessive or insufficient growth of new blood vessels. Excessive blood vessel proliferation caused by over-production of angiogenesis growth factors can lead to enhanced tumour growth and spread, or cause rheumatoid arthritis, psoriasis or diabetic blindness. In fact, there are some 70 known conditions that result from so-called “excessive angiogenesis”. In many of these conditions, new blood vessels feed diseased tissue, destroy normal tissue, and, in the case of cancer, the new vessels allow tumour cells that have spread to other tissue through blood circulation to become established (i.e. tumour metastases).

In contrast, conditions such as coronary artery disease, ulcers, stroke and delayed wound healing result from “insufficient angiogenesis” because the tissue is not able to produce adequate amounts of angiogenesis growth factors. Angina or chest pain, for example, occurs because of a decreased supply of blood to the heart. In these conditions there are inadequate blood vessels to restore proper blood circulation leading to the risk of tissue death.

Prolonged wound healing and ulcer are also caused by insufficient angiogenesis. Wound healing is a natural restorative response to tissue injury and involves the interaction of a complex cascade of cellular events that can be described in terms of 3 classic phases, namely inflammation, proliferation and maturation (or remodelling). Angiogenesis occurs during the proliferative phase of wound healing to facilitate repair of the vasculature tissue surrounding the wound, and to increase the supply of nutrients from the blood stream in order to stimulate the local cell activity necessary for healing. Moreover, the endothelial cells which form the lining of blood vessels are important organisers and regulators of the tissue healing response.

Various methods of modulating wound healing are known. In particular, the use of certain plant extracts is known to promote wound healing. Such plant extracts include non-fruit plant extracts as well as extracts from fruits, both citrus and non-citrus fruits.

EP 0210785 describes a proanthocyanide A2 extract derived from the pericarp, cortex or branches of Aesculus hippocastanum (Horse Chestnut) which was shown to enhance wound healing activity in both rats and mice. U.S. Pat. No. 4,587,124 describes a heat-treated oil extract of Strychnos ignatii Berg (forest vine) and method for treating cutaneous wounds by administering the extract.

One example of a citrus fruit extract with biological activity is described in DE 19929298. The use of an adhesive plaster or pad treated with a grapefruit flesh and/or seed extract is described. The grapefruit extract is described as having bactericidal, fungicidal, anti-viral and anti-parasitic activity leading to enhanced wound healing and reduced scarring. WO 2004/091569 describes an extract of citrus fruit peel that has been “activated” by a plant or animal pathogen, such as a fungal or bacterial pathogen. The usefulness of this extract appears to be limited to the treatment of skin conditions.

Non-citrus fruits are also known to promote wound healing. For example, RU 2140254 describes a cosmetic liquid comprising an extract of juniper fruit for accelerated wound healing. JP 2124809 describes a cosmetic formulation containing an extract from reishi fruit also used to enhance wound recovery. Yet another example is CN 1103802 which relates to a suppository containing a cnidium fruit extract to promote tissue regeneration and wound healing.

There is, however, an ongoing search for improved angiogenesis promoting substances. Many of the known plant extracts that promote angiogenesis are difficult to obtain, have undesirable impurities, and may be costly to produce. Further, most plant extracts that have an effect on angiogenesis are extracts that inhibit angiogenesis. Extracts that promote angiogenesis are less common and are therefore of much interest. There is also the problem with fruit extracts that because typically whole fruit (including skin, flesh and seeds) are extracted, complex mixtures containing undesirable impurities are usually obtained.

The applicant has now surprisingly found that extracts of the skin of citrus fruit have wound healing and/or angiogenesis promoting activity.

It is therefore an object of the invention to provide the use of an extract of the skin of citrus fruit in therapies related to angiogenesis promotion or wound healing, or to at least provide a useful choice.

### STATEMENTS OF INVENTION

In a first aspect, the invention provides a method of promoting angiogenesis, or treating a disease or disorder where it is desirable to promote angiogenesis, comprising administering a therapeutically effective amount of an extract of the skin of a citrus fruit to a human or non-human animal.

Preferably the disease or disorder is muscle ischemia, stroke, or ulcer. More preferably the muscle ischemia is skeletal muscle ischemia or is caused by coronary artery disease.

The promotion of angiogenesis may be to assist wound healing. The promotion of angiogenesis may alternatively be in engineered or regenerated tissues in tissue culture.

Preferably, the citrus fruit is grapefruit, orange, lemon, lime, mandarin, tangelo, tangerine, or uglis. In a preferred embodiment of the invention, the citrus fruit is grapefruit (Citrus paradisii).

Preferably, for the treatment of external wounds the extract is administered topically in the form of a gel, spray, cream or lotion. Alternatively, the extract may be administered orally, intraperitoneally, intravenously, subcutaneously, intramuscularly, or in any other suitable manner.

In a second aspect, the invention provides the use of a therapeutically effective amount of an extract of the skin of a citrus fruit in the manufacture of a medicament for promoting angiogenesis, or treating a disease or disorder where it is desirable to promote angiogenesis.

In another aspect, the invention provides a composition comprising an extract of the skin of a citrus fruit suitable for use in promoting angiogenesis, or treating a disease or disorder where it is desirable to promote angiogenesis.

In a further aspect, the invention provides a process for preparing an extract of the skin of a citrus fruit by:
(a) homogenising the skin of the citrus fruit and adding to the resulting homogenate about 5 volumes of an aqueous solvent;
(b) stirring the mixture obtained in step (a);
(c) filtering the mixture obtained in step (b) to give a filtrate;
(d) concentrating the filtrate at about 45° C. under reduced pressure;
(e) subjecting the concentrated filtrate to chromatographic purification where, following elution with water, a fraction obtained by elution with an alcohol is obtained;
(f) concentrating the alcohol fraction at about 45° C. under reduced pressure to give the extract.

The steps of stirring the mixture and filtration may optionally be repeated to maximise the extraction from the skin.

The chromatographic purification of the concentrated filtrate is preferably carried out by adsorbing the concentrated filtrate onto a non-ionic resin chromatography medium, eluting firstly with water to remove undesirable material, then eluting with an alcohol to give a faction containing the extract.

The solvent used in the aqueous solvent of step (a) may be selected from the group consisting of methanol, ethanol, propanol and acetone.

Preferably, the solvent used in the aqueous solvent of step (a) is 70% aqueous acetone. It is also preferred that the non-ionic resin used in step (e) is polystyrene. It is further preferred that the adsorbed fraction is eluted from the column in step (f) using methanol.

### BRIEF DESCRIPTION OF THE FIGURES

FIGS. 1A to 1I compare two substantially identical circular excision wounds inflicted in the back of a Lewis rat over a 17 day period. The left wound has been treated with 25 μl of grapefruit skin extract (10 mg/ml) every second day.

FIGS. 2A to 2I also compare two substantially identical circular excision wounds inflicted in the back of a Lewis rat over a 17 day period. This time, the left wound has been treated with 25 μl of grapefruit skin extract (1 mg/ml) every second day.

FIG. 3 compares the mean wound size of the treated and untreated wounds as a function of time after wounding. The treated wounds received 25 μl of grapefruit skin extract (10 mg/ml) every second day. The comparison is expressed as a percentage of the original wound area. For each point n=5.

FIG. 4 compares the mean wound size of the treated and untreated wounds as a function of time after wounding. The treated wounds received 25 μl of grapefruit skin extract (1 mg/ml) every second day. The comparison is expressed as a percentage of the original wound area. For each point n=5.

### DETAILED DESCRIPTION

As described herein, “skin” of a citrus fruit means the remaining part of the citrus fruit after the edible pulp and seed inside has been removed.

The invention relates to an extract of the skin of a citrus fruit, which extract has angiogenesis promoting activity. The extract is therefore useful in treating diseases or disorders where it is desirable to promote angiogenesis, such as coronary artery disease, stroke or ulcer. The extract is also useful in assisting wound healing. Furthermore, the extract may be used for the promotion of angiogenesis in tissue culture, as in engineered or regenerated tissues in extracellular matrices for tissue graftings. In tissue culture, the extract can promote a vascular supply that can support the needs of engineered or regenerated tissues in extracellular matrices.

Advantageously, the active extract is obtained from the skin of citrus fruit, which is often a waste product of commercial citrus fruit processing. Further, the process for obtaining the extract from skin of citrus fruit is straightforward. The extract of the skin also has the advantage of having fewer undesirable impurities than a whole fruit extract.

The invention is described in detail with regard to grapefruit as the preferred citrus fruit of the invention. However, it is to be appreciated that the invention relates to any citrus fruit including, but not limited to, grapefruit, orange, lemon, lime, mandarin, tangelo, tangerine, and uglis.

An in vitro culture assay for quantitative evaluation of the rate of angiogenesis in rat aorta was employed in order to determine the effect of the grapefruit skin extract on angiogenesis (Example 2). The area of microvessel growth relative to the aortic ring for tissue treated with the grapefruit skin extract resulted in an increase in new blood vessel formation of 381% compared to the control. This result clearly demonstrates the effectiveness of the grapefruit skin extract for promoting angiogenesis.

To examine the effect of the grapefruit skin extract on wound healing, the rate of wound recovery (measured as the total wound area) was determined for Lewis rats inflicted with substantially identical circular excision wounds. One wound served as a test wound, and the other as a control. Two separate experiments were performed in which the dose of the grapefruit skin extract was 10 mg/ml and 1 mg/ml.

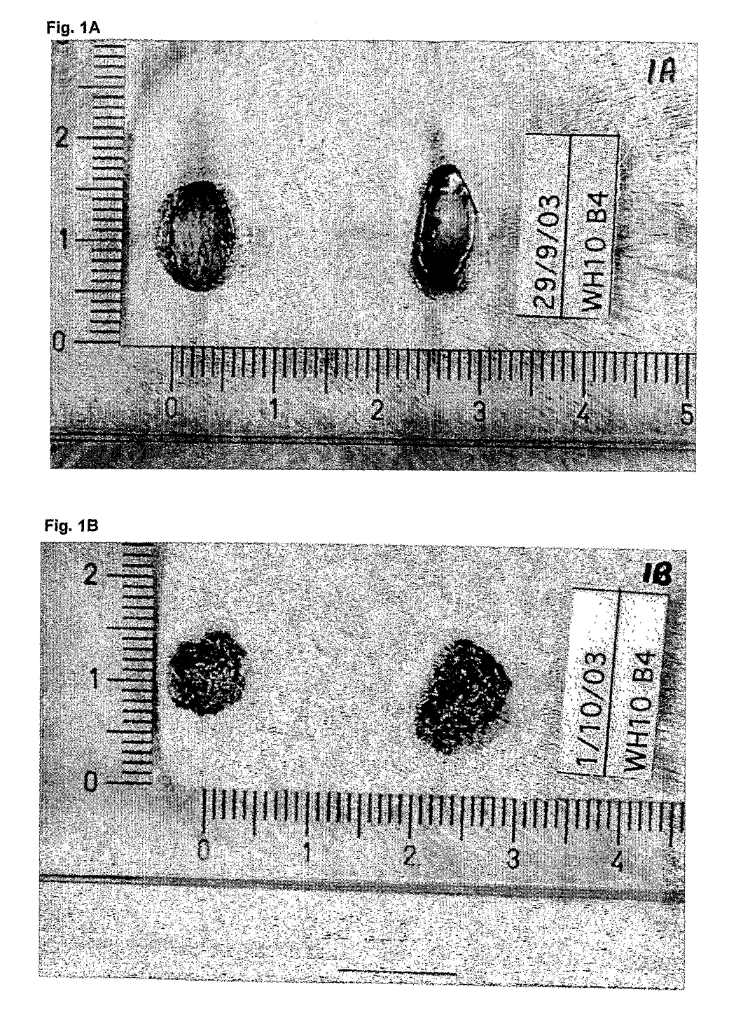

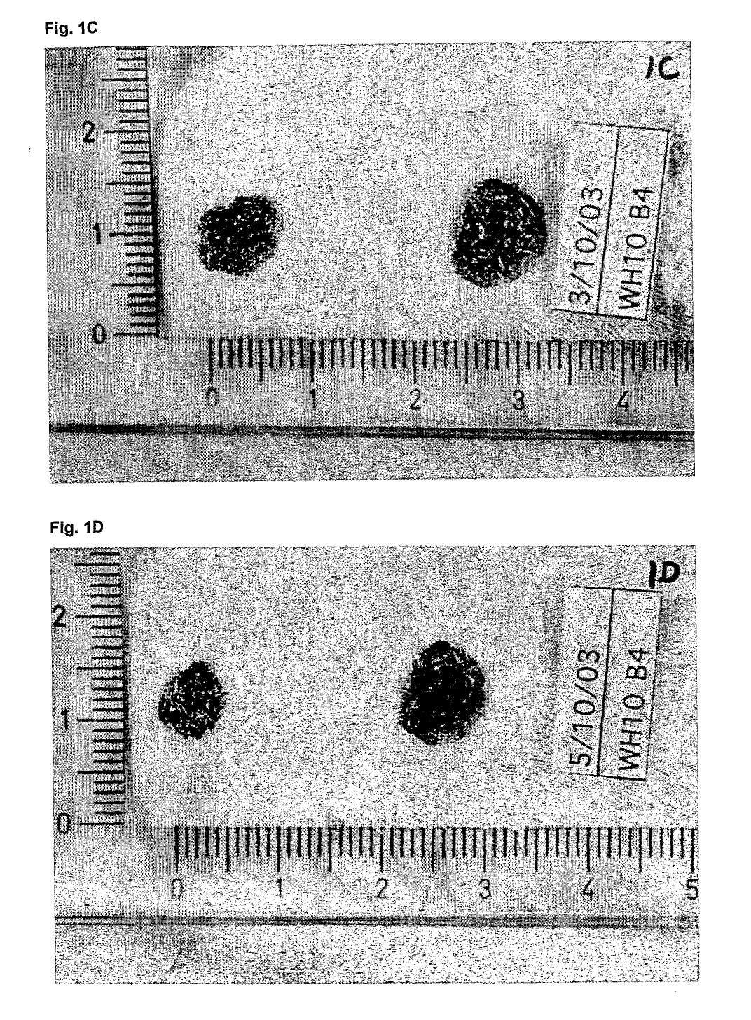

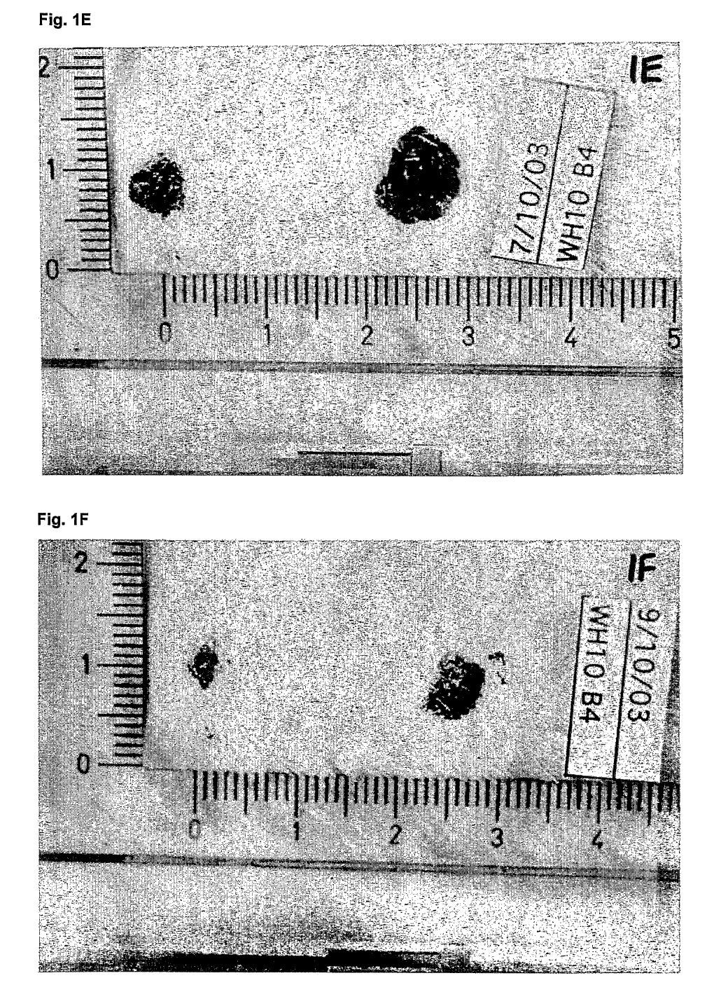

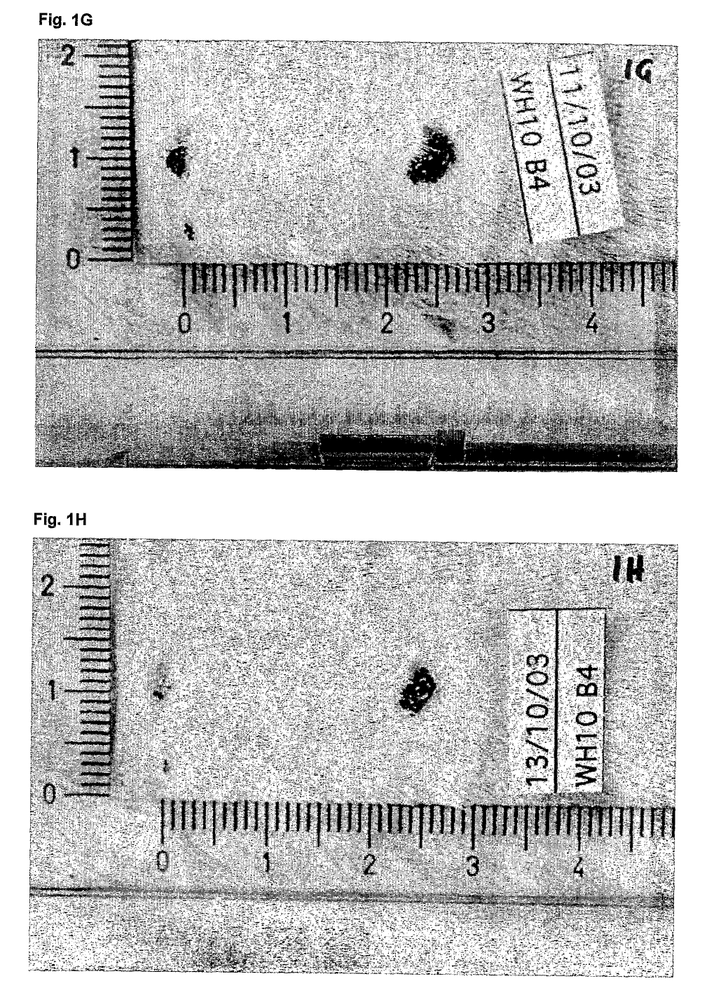

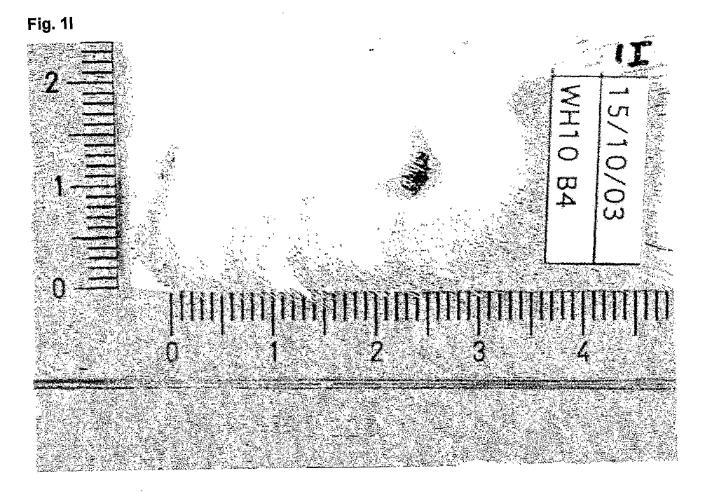

For the first experiment, the grapefruit skin extract was administered topically at a dose of 10 mg/ml to the test wound (left side wound) every second day over a 17 day trial (FIGS. 1A to 1I). FIG. 1A shows the test and control wounds immediately following wound infliction. FIGS. 1B-1I show the identical wounds at two day intervals following infliction, up to day 17 (FIG. 1I). Clearly, the test wound has healed significantly faster than the control.

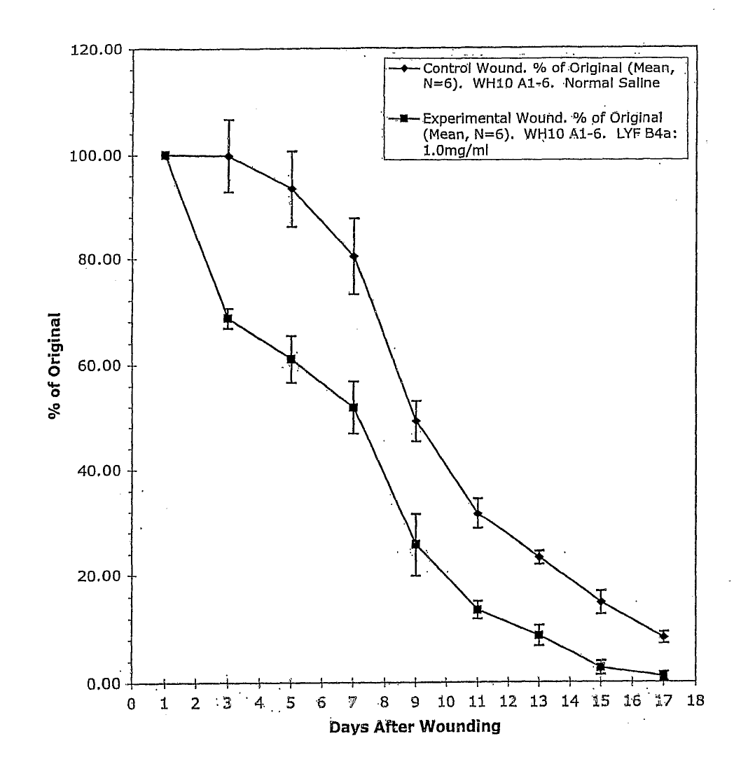

To quantify the relative rate of wound recovery the total wound area for each wound was measured. The data are presented in Table 1 and FIG. 3 as a total percentage of the original wound area over time. In Table 1, the ratios of the total wound area for the test and control wounds are also given. Because the ratio is less than 1.00 at each time point, the implication is that the treated wound is healing faster than the control wound. There is a significant acceleration of the decrease in wound surface area over the first 5 days (FIG. 3). It is of interest to note that the angiogenic phase of wound healing predominantly occurs in this period.

TABLE 1

| | Experimental wound | Control wound area | Ratio |
|---|---|---|---|
| | area (mm² ± SEM) | (mm² ± SEM) | (±SEM) |
| Day 1 | 100.00 ± 0.00 | 100.00 ± 0.00 | 1.00 |
| Day 3 | 63.73 ± 1.15 | 97.07 ± 2.88 | 0.66 |
| Day 5 | 59.11 ± 2.88 | 99.44 ± 7.14 | 0.59 |
| Day 7 | 48.85 ± 2.84 | 89.43 ± 8.07 | 0.55 |
| Day 9 | 23.79 ± 3.09 | 48.92 ± 7.55 | 0.52 |
| Day 11 | 18.34 ± 3.42 | 34.86 ± 3.68 | 0.53 |
| Day 13 | 11.60 ± 1.41 | 23.98 ± 2.84 | 0.48 |
| Day 15 | 3.21 ± 1.18 | 14.71 ± 1.95 | 0.22 |
| Day 17 | 0.31 ± 0.15 | 7.16 ± 2.28 | 0.04 |

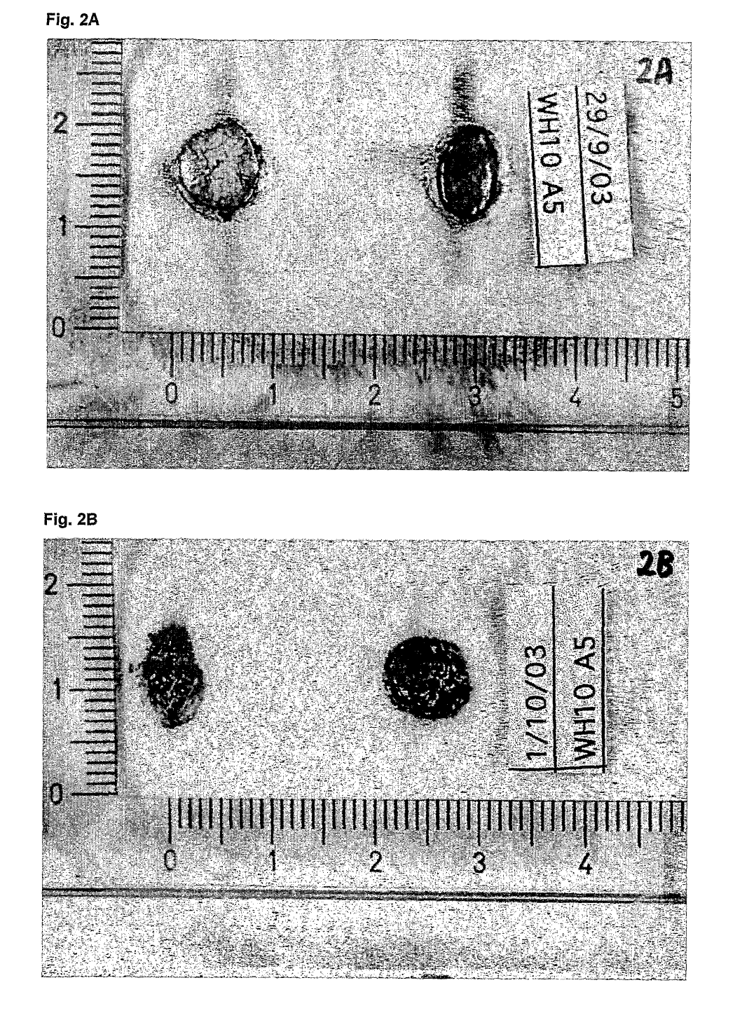

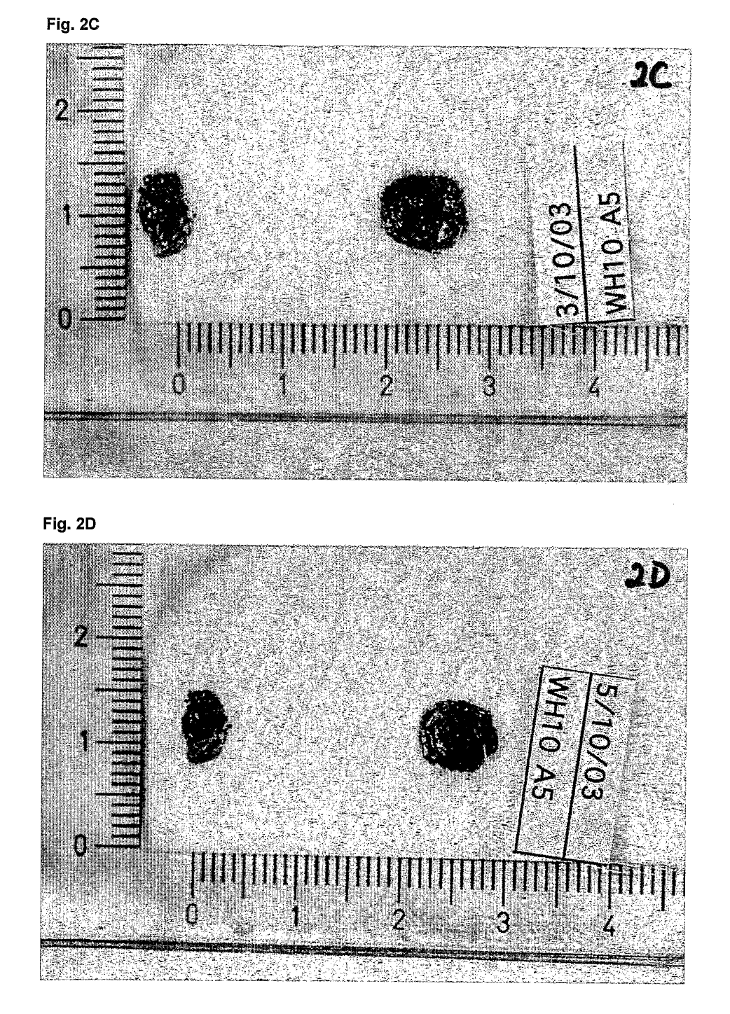

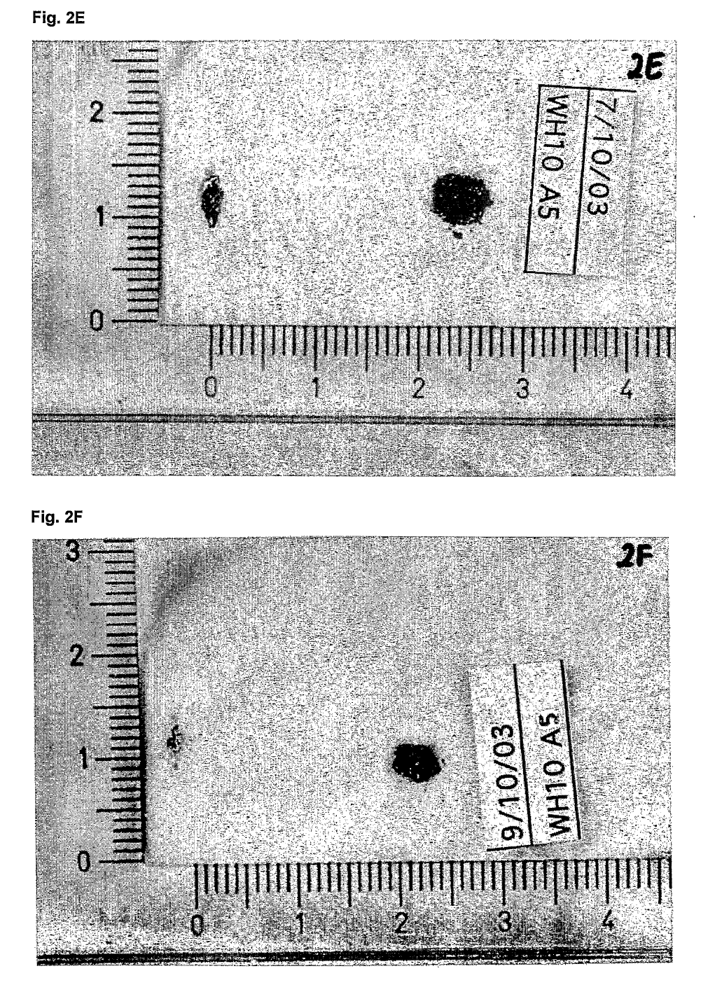

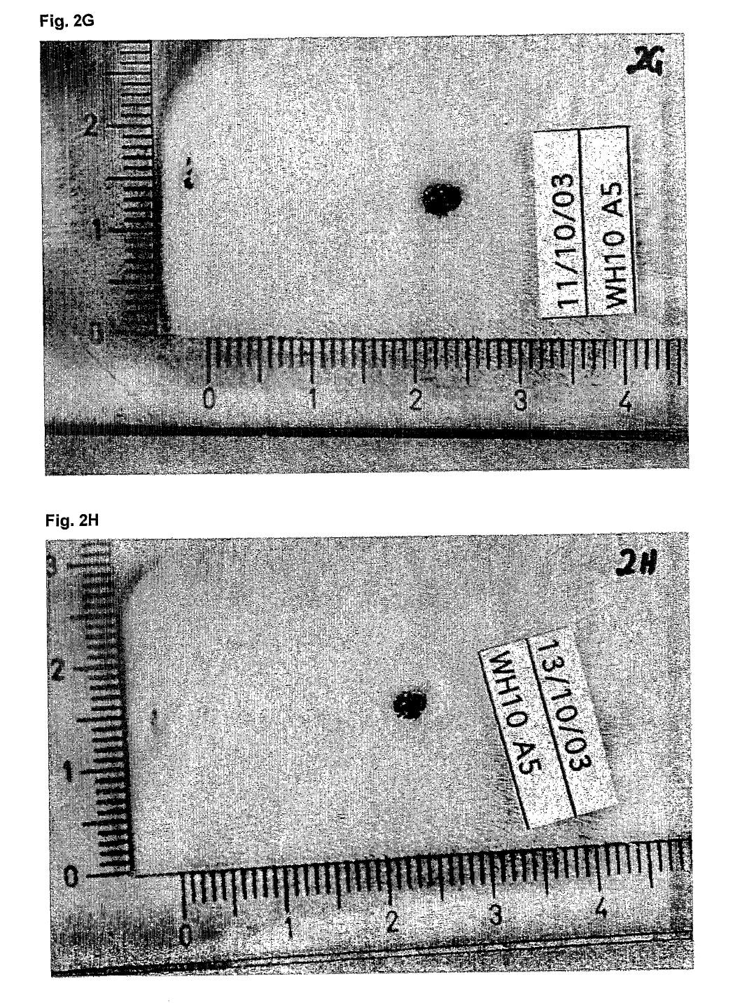

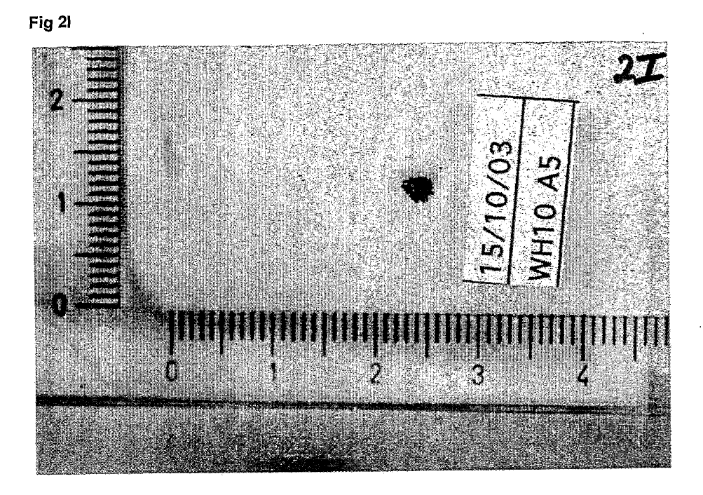

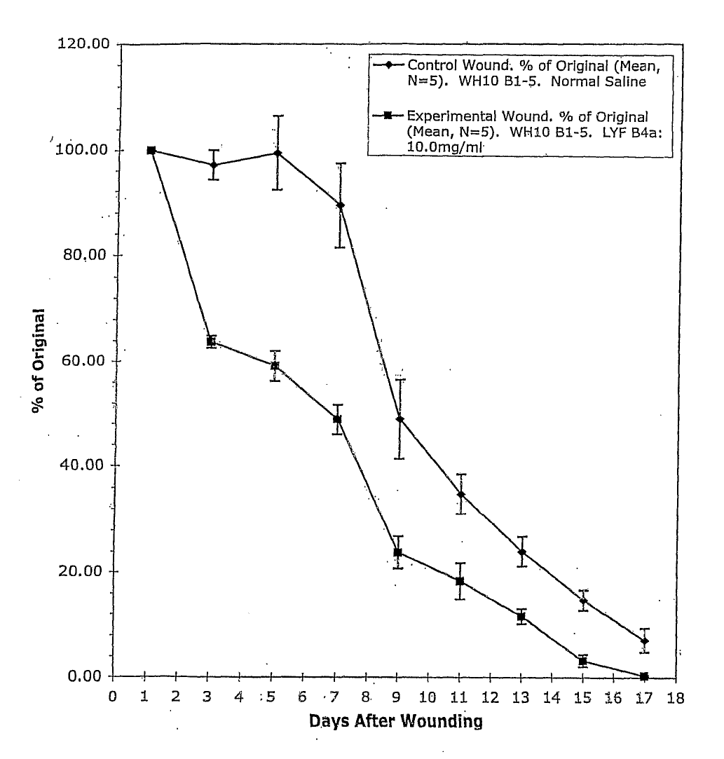

For the second experiment, the grapefruit skin extract was administered topically at a dose of 1 mg/ml. FIG. 2A shows the test and control wounds immediately following wound infliction. FIGS. 2B-2I show the identical wounds at two day intervals following infliction, up to day 17 (FIG. 2I). Again, the test wound has healed significantly faster than the control.

As with the first experiment, a measure of the total wound area (expressed as a percentage of the original wound area) for rats administered with 1 mg/ml of grapefruit skin extract compared to the control is presented in Table 2 and FIG. 4. Even at the lower concentration, the grapefruit skin extract has a very similar effect on the overall rate of wound recovery.

TABLE 2

| | Experimental wound | Control wound area | Ratio |
|---|---|---|---|
| | area (mm² ± SEM) | (mm² ± SEM) | (±SEM) |
| Day 1 | 100.00 ± 0.00 | 100.00 ± 0.00 | 1.00 |
| Day 3 | 68.50 ± 1.83 | 99.70 ± 6.93 | 0.69 |
| Day 5 | 60.95 ± 4.33 | 93.35 ± 7.23 | 0.65 |
| Day 7 | 51.79 ± 5.03 | 80.29 ± 7.31 | 0.65 |
| Day 9 | 25.80 ± 5.86 | 49.18 ± 3.88 | 0.52 |
| Day 11 | 13.59 ± 1.64 | 31.68 ± 2.88 | 0.43 |
| Day 13 | 8.80 ± 1.96 | 23.33 ± 1.19 | 0.38 |
| Day 15 | 2.76 ± 1.32 | 14.96 ± 2.13 | 0.18 |
| Day 17 | 1.11 ± 0.85 | 8.38 ± 1.11 | 0.13 |

It will be appreciated by those skilled in the art that the citrus fruit skin extract may be administered topically in the form of a gel, spray, cream or lotion. The extract may also be administered orally, intraperitoneally, intravenously or in any other suitable manner depending on the disease or disorder to be treated.

The amount of extract to be administered will vary widely depending upon the patient and the nature and extent of the disorder to be treated.

The applicant has found that the citrus fruit skin extract is advantageously prepared from fruit skin, which is often a waste product of commercial citrus fruit processing. After pulverising, the fruit skin is extracted in 70% aqueous solvent, such as 70% aqueous acetone. The solvent is removed, and purification is readily effected by chromatography.

Elution with an alcohol, such as methanol, gives a fraction which can be concentrated, taken up with water, and then lyophilized to give the desired extract.

The invention is further described with reference to the following examples. However, it is to be appreciated that the invention is not limited to these examples.

### EXAMPLES

### Example 1: Extraction Procedure

The peelings from citrus fruits were pulverized in an electric blender and to this material was added 5 volumes of 70% aqueous acetone (acetone/water 7:3 v/v). The mixture was stirred for 2 hours and then left to stand at ambient temperatures overnight. The resulting mixture was filtered over nylon cloth, squeezing the pulp while doing so. The solid residue was re-suspended in aqueous solvent, stirred for an hour and then filtered. The filtrates were combined and the acetone was removed by concentration at 45° C. under reduced pressure. The residual aqueous fraction was decanted for collection and treated over column of non-ionic polystyrene resins. The column was washed in 3 volumes of distilled water and the absorbed materials eluted with methanol and concentrated under reduced pressure. The residual material was diluted with 1 volume of distilled water lyophilized to give the active extract.

### Example 2: Angiogenesis Assay

After the removal of fat and perivascular fibrous tissue, rat aorta was cut into rings of about 2 mm thickness. A plug of fibrin gel (0.4 ml), prepared by adding thrombin to fibrinogen solution dissolved in MCDB 131 medium, was formed in a well of a 24-well culture plate. An aorta ring was placed in the centre of each well and overlaid with another plug of fibrin. Each gel was covered with MCDB 131 medium (1.5 ml) and incubated at 37° C. in an atmosphere of 3% CO₂/97% air. Samples to be tested were added as supplements to the medium. Each sample was assayed in triplicate.

After approximately 5 days, microvessels could be detected growing from the perimeter of the rings. At regular intervals between 5 to 14 days, images of each well were recorded using a digital camera attached to an inverted microscope. The area of microvessel growth relative to the perimeter of the ring for each image was measured at each time point. A mean value for the growth rate was determined and the rate of microvessel was then calculated for each sample.

### Example 3: Wound Healing

Two circular excisional wounds were established in the back of each rat using a 0.8 cm skin biopsy punch just below the shoulders. One wound was inflicted on each side of the mid-line. Two doses of the test material were studied. For each experiment 5 male Lewis rats were used. Aliquots (25 μl) of the grapefruit skin extract dissolved in normal saline were applied topically to the left wound of each animal every second day. For one group of rats the concentration was 10 mg/ml and for the second group it was 1 mg/ml. Vehicle was added to the contralateral wound to serve as a control. Photographs of each pair of wounds were taken prior to each addition using a Canon EOS 3000 N camera (F2.8 Macro lens) and Fuji Professional 40ONPH film. Prints of each exposure were recorded digitally and the area of each wound calculated from these images using NIH Image 1.63 software. The results can be visualised in FIGS. 1 and 2.

Although the invention has been described by way of example, it should be appreciated that variations and modifications may be made without departing from the scope of the claims. Furthermore, where known equivalents exist to specific features, such equivalents are incorporated as if specifically referred to in the specification.

### INDUSTRIAL APPLICABILITY

The invention enables the promotion of angiogenesis. This has applicability to the promotion of wound healing as well as the treatment of a variety of diseases or disorders, including coronary artery disease, stroke, and ulcer. A process for obtaining the citrus fruit skin extract used in the invention involves extracting the skin of the citrus fruit with an aqueous solvent, and purifying a filtrate followed by solvent removal to give the extract.

1. A method of promoting angiogenesis in a human or non-human animal, comprising:
identifying a human or non-human animal in need of a promotion of angiogenesis; and
providing said human or non-human animal a therapeutically effective amount of an extract of the skin of a citrus fruit.

2. The method as claimed in claim 1, wherein said human or non-human animal in need of a promotion of angiogenesis has muscle ischemia, stroke, or ulcer.

3. The method as claimed in claim 2 wherein the muscle ischemia is a skeletal muscle ischemia or an ischemia caused by coronary artery disease.

4. The method as claimed in claim 1 wherein the extract of the skin of said citrus fruit is provided to a wound.

5. (canceled)

6. The method as claimed in claim 1, wherein the citrus fruit is grapefruit, orange, lemon, lime, mandarin, tangelo, tangerine, or uglis.

7. The method as claimed in claim 1, wherein the citrus fruit is grapefruit (Citrus paradisii).

8. The method as claimed in claim 1, wherein the extract is provided orally, intraperitoneally, intravenously, subcutaneously or intramuscularly.

9. The method as claimed in claim 1, wherein, the extract is provided in a gel, spray, cream or lotion.

10. (canceled)

11. (canceled)

12. (canceled)

13. (canceled)

14. A composition comprising a therapeutically effective amount of an extract of the skin of a citrus fruit formulated for administration to a human or non-human animal.

15. A process for preparing an extract of the skin of a citrus fruit comprising:
(a) homogenizing the skin of the citrus fruit and adding to the resulting homogenate about 5 volumes of an aqueous solvent;
(b) stirring the mixture obtained in step (a);
(c) filtering the mixture obtained in step (b) to give a filtrate;
(d) concentrating the filtrate at about 45° C. under reduced pressure;
(e) subjecting the concentrated filtrate to chromatographic purification where, following elusion with water, a fraction obtained by elusion with an alcohol is obtained; and
(f) concentrating the alcohol fraction at about 45° C. under reduced pressure to give the extract.

16. The process as claimed in claim 15, wherein the steps of stirring the mixture and filtration are repeated.

17. The process as claimed in claim 15, wherein the chromatographic purification of the concentrated filtrate is carried out by adsorbing the concentrated filtrate onto a non-ionic resin chromatography medium, eluting firstly with water to remove undesirable material, then eluting with an alcohol to give a faction containing the extract.

18. The process as claimed in any one claim 15, wherein the solvent used in the aqueous solvent of step (a) is selected from the group consisting of methanol, ethanol, propanol, and acetone.

19. The process as claimed in claim 15, wherein the solvent used in the aqueous solvent of step (a) is 70% aqueous acetone.

20. The process as claimed in claim 15, wherein the non-ionic resin used in step (e) is polystyrene.

21. The process as claimed in claim 15, wherein the adsorbed fraction is eluted from the column in step (f) using methanol.

22. A method of promoting angiogenesis, comprising:
providing an engineered or regenerated tissue in cell culture; and
contacting said engineered or regenerated tissue with an amount of an extract of the skin of a citrus fruit sufficient to promote angiogenesis.

23. The method of claim 22, further comprising measuring angiogenesis.

24. The method of claim 1, further comprising measuring angiogenesis in said human or non-human animal.

25. The composition of claim 14, wherein said composition is a topical formulation.

26. The composition of claim 14, wherein said composition is a gel, spray, cream, or lotion.

27. A method of accelerating the healing of a wound, comprising:
identifying a subject in need of acceleration of the healing of a wound; and
providing said subject a topical composition formulated for administration to a human or non-human animal that comprises an extract of the skin of a citrus fruit.

28. The method of claim 27, further comprising monitoring the healing of the wound.

29. The method of claim 28, wherein said wound is an ulcer.
